(19) United States
(12) Patent Application Publication
(10) Pub. No.: US 2013/0289578 A1
(43) Pub. Date: Oct. 31, 2013

(54) BLOOD CLOT EXTRACTION DEVICE

(71) Applicants: GERARDO V. NORIEGA, MOUNTAIN VIEW, CA (US); RODOLFO B. SUDARIA, UNION CITY, CA (US); SARAH E. VAN KEULEN, MORGAN HILL, CA (US); SOFIA A. POUR, SARATOGA, CA (US); KRISTIAN C. HOCKENSON, EL DORADO HILLS, CA (US); VICTOR CHECHELSKI, MOUNTAIN VIEW, CA (US)

(72) Inventors: GERARDO V. NORIEGA, MOUNTAIN VIEW, CA (US); RODOLFO B. SUDARIA, UNION CITY, CA (US); SARAH E. VAN KEULEN, MORGAN HILL, CA (US); SOFIA A. POUR, SARATOGA, CA (US); KRISTIAN C. HOCKENSON, EL DORADO HILLS, CA (US); VICTOR CHECHELSKI, MOUNTAIN VIEW, CA (US)

(73) Assignee: YAWA-MED, INC., MOUNTAIN VIEW, CA (US)

(21) Appl. No.: 13/859,083

(22) Filed: Apr. 9, 2013

Related U.S. Application Data
(60) Provisional application No. 61/686,767, filed on Apr. 13, 2012.

(51) Int. Cl. A61B 17/221

(52) U.S. Cl. CPC A61B 17/221
USPC 606/127

(57) ABSTRACT

A device for capturing a clot in a blood vessel comprises an outer shaft, an inner shaft slidably disposed in a lumen of the outer shaft, and clot capture arms secured to a distal end of the inner shaft. The device is navigated and advanced through the vasculature to reach the location of a blood clot. An anchoring element on the outer shaft is expanded to anchor the device at the location of the blood clot. The clot capture arms are extended from the open end of the outer shaft. As the clot capture arms are extended, the distal portions of the arms gradually open into radially inwardly curved configurations to capture the clot. With the clot captured, the arms are retracted back into the outer shaft along with the clot. The anchoring element is then collapsed, and the device, with the clot captured therein, is retracted from the vasculature.

### CROSS-REFERENCE

This application claims the benefit of U.S. Provisional Application No. 61/686,767, filed Apr. 13, 2012, the contents of which are incorporated herein by reference.

### BACKGROUND OF THE INVENTION

The present disclosure relates to medical devices, systems, and methods. In particular, the present disclosure relates to medical devices, systems, and methods for treating or preventing pulmonary embolism in a minimally invasive manner.

In the United States, more than 600,000 people have a pulmonary embolism each year, and more than 60,000 of them die. Pulmonary embolism occurs when a blood clot, or thrombus detaches from a larger embolus located somewhere else in the body, and travels through the venous system into the pulmonary trunk where it becomes ledged, blocking blood flow. This foreign body does not allow for the blood to flow into the lungs. Consequently, blood supply to the lungs, heart, and potentially the brain are inhibited. Such inhibited blood supply could ultimately result in a patient experiencing difficulty breathing, palpitations, stroke, or even death. Pulmonary embolisms commonly affect those who experience poor circulation, especially in their legs, such as frequent airplane travelers, those bedridden due to recent surgery or ailment, cancer patients, and even people taking certain medications such as birth control.

There are many current therapies and surgical procedures that aim to treat or remove blood clots. However, many therapies and devices used in the treatment as well as surgery can pose problems including severe bleeding or hemorrhaging, reformation of emboli in another location, and even anemia resulting from possible hemolysis. Therefore, improved therapies and devices for treating and preventing pulmonary embolism are desired.

### SUMMARY OF THE INVENTION

The present disclosure provides a minimally invasive approach to restore blood flow to the limbs and organs, and more specifically to the venous and arterial sites. Blood clots are removed from the human anatomy, specifically in the venous and arterial system and more specifically in the lungs and the peripheral vasculature. The present disclosure provides an approach that is intuitively easy to use, making it a preferred choice for a physician in the treatment of pulmonary embolism and venous and arterial blockages.

An aspect of the disclosure provides a device for capturing clot in a blood vessel. The device comprises an outer shaft, an inner shaft, and a plurality of clot capture arms. The inner shaft is slidably disposed in a lumen of the outer shaft. The plurality of clot capture arms is secured to a distal end of the inner shaft. At least some of the arms have radially inwardly curved distal portions. The arms are configured to open as the distal portions are advanced from the outer shaft and close as the distal portion is retracted into the outer shaft.

Both the outer and inner shafts may be flexible, torquable, and comprise at least one marker adjacent their distal ends. The inner shaft will typically be rotatable within the lumen of the outer shaft.

The device may further comprise a proximal hub coupled to a proximal end of the inner shaft. The proximal hub comprises a rotatable element rotatable in a first direction. Rotating the rotatable element in the first direction can causes the inner shaft to rotate in a second direction opposite the first direction. The inner shaft may be threaded such that rotation of the inner shaft causes a thread of the inner shaft to carry at least a portion of a captured clot in a proximal direction to remove the clot. The tubular inner shaft may comprise a reverse thread. The proximal hub provide other functions as well, including diverting captured and removed clot away from the proximal end of the outer shaft and the proximal end of the inner shaft.

The plurality of arms may be configured in many ways. Typically, they are biased to open. The arms may be configured to adjust to a size of the blood vessel when open and advanced from the outer shaft. The arms may be shaped to reduce vessel trauma when open and advanced from the outer shaft. At least some of the arms of the plurality of arms have an arcuate shape when open and advanced from the outer shaft. There may be various numbers of arms in various embodiments. For example, the plurality of arms may comprise at least one pair, at least two pairs, at least three pairs, or even more of diametrically opposed arms. The arms may be made of a shape memory material such as nitinol or may be made of another resilient material such as stainless surgical steel. The plurality of arms may comprise at least one connector coupling an arm of the plurality of arms to an adjacent arm. Such a connection may be made at the proximal portions of the arms. The connector may be a U-shaped connector, for example, a U-shaped connected wherein the open end of the U-shaped connector faces proximally and the closed end of the U-shaped connector faces distally.

The device may further comprise an expandable element adjacent the distal end of the outer shaft. The expandable element is expandable to anchor the outer shaft within the blood vessel near the clot. The expandable element may be an inflatable balloon, and the outer shaft may further comprise an inflation lumen for inflating and deflating the inflatable expandable element.

Suction or negative pressure may also be applied to facilitate the extraction of a clot or clot material. The device may further comprise a suction element coupled to the proximal hub of the device to apply suction through an inner lumen of the inner shaft and/or the inner lumen of the outer shaft.

Another aspect of the disclosure provides a method for treating a pulmonary embolism by capturing clot in a pulmonary artery or peripheral vein. A catheter is introduced into a location in the pulmonary artery or peripheral vein proximal of the clot. At least distal portions of a plurality of clot capture arms are advanced out from an open end of the catheter to surround at least a portion of the clot. These distal portions are retracted back into the catheter to capture the portion of the clot. An expandable element near the open end of the outer shaft may be expanded to anchor the outer shaft near the clot in the pulmonary artery or peripheral vein. The expandable element may be expanded by inflating it through an inflation lumen of the outer shaft.

Often, at least a portion of the captured clot may be broken, for example, by rotating the plurality of clot capture arms against the clot or by advancing the plurality of clot capture arms against the clot. The plurality of clot capture arms can be rotated by rotating an inner shaft slidably disposed within the outer shaft. The inner shaft typically has a distal end coupled to a proximal end of the plurality of clot capture arms. Rotating the inner shaft can also causes a thread of the inner shaft to carry the portion of the clot in a proximal direction within the outer shaft to remove the portion of the clot. The inner shaft can be rotated by rotating a rotatable element of a hub coupled to a proximal end of the inner shaft to rotate the distal end of the inner shaft. The rotatable element may be rotated in a first direction to rotate the distal end of the inner shaft in a second direction opposite the first direction. The captured portion of clot may be diverted away from the proximal end of the outer shaft. Suction or negative pressure may be applied through the inner lumen of the outer shaft and/or the inner lumen of the inner shaft to remove the captured portion of clot.

Advancement of the plurality of clot capture arms from the open end of the outer shaft typically opens the clot capture arms as the arms are advanced from the open end of the outer shaft. The plurality of arms is typically shaped to reduce vessel trauma when advanced out of the open end of the catheter. At least some of the arms of the plurality of clot capture arms have radially inwardly curved distal portions when advanced out of the open end of the catheter. At least some of the arms of the plurality of clot capture arms have an arcuate shape when advanced out of the open end of the catheter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a side view of a blood clot extraction device according to an embodiment of the disclosure;

FIG. 1B is a cross-sectional side view of the blood clot extraction device of FIG. 1A;

FIG. 1C is a side view of the blood clot extraction device of FIG. 1A having its blood clot capture arms partially extended and its anchoring balloon expanded;

FIG. 1D is a side view of the blood clot extraction device of FIG. 1A having its blood clot capture arms fully extended and its anchoring balloon expanded;

FIGS. 1E and 1F are side views of the blood clot extraction device of FIG. 1A having its blood clot capture arms rotated;

FIG. 1G is a cut away side view of the outer shaft and threaded inner shaft of the blood clot extraction device of FIG. 1A;

FIG. 2A is a cross-sectional view of the blood clot extraction device of FIG. 1A taken along line 2 in FIG. 1A;

FIG. 2B is a cross-sectional view of a blood clot extraction device according to another embodiment of the disclosure;

FIG. 2C1 is a cross-sectional view of a blood clot extraction device according to yet another embodiment of the disclosure;

FIG. 2C2 is a cross-sectional side view of the blood clot extraction device of FIG. 2C1;

FIG. 2D1 is a cross-sectional view of a blood clot extraction device according to a further embodiment of the disclosure;

FIG. 2D2 is a cross-sectional view of the blood clot extraction device of FIG. 2D1;

FIG. 3 is a cross-sectional view of the blood clot extraction device of FIG. 1A taken across line 3 in FIG. 1A;

FIG. 4A is a cross-sectional view of a blood clot extraction device having an inner shaft with a solid core according to a yet further embodiment of the disclosure;

FIG. 4B1 shows a cut-away side view of a blood clot extraction device having an inner shaft comprising a plurality of wound cables according to an even further embodiment of the disclosure;

FIG. 4B2 shows a cross-sectional view of the inner shaft of the blood clot extraction device of FIG. 4B1;

FIG. 5A shows a side view of blood clot capture arms according to many embodiments of the disclosure;

FIG. 5B shows an end view of the blood clot capture arms of FIG. 5A;

FIG. 5C shows a side view of the blood clot capture arms of FIG. 5A rolled out and flattened;

FIG. 5D shows a side view of blood clot capture arms rolled out and flattened according to other embodiments of the disclosure; and

FIGS. 6A to 6F show side views of a method of extracting a blood clot according to an embodiment of the disclosure.

### DETAILED DESCRIPTION OF THE INVENTION

Devices, systems, and methods providing a minimally invasive approach to restore blood flow to the limbs and organs, and more specifically to the venous and arterial sites, are now described. Blood clots are removed from the human anatomy, specifically in the venous and arterial system and more specifically in the lungs and the peripheral vasculature. The devices, systems, and methods described herein are intuitively easy to use, making them a preferred choice for physicians in the treatment of pulmonary embolism, and venous and arterial blockages.

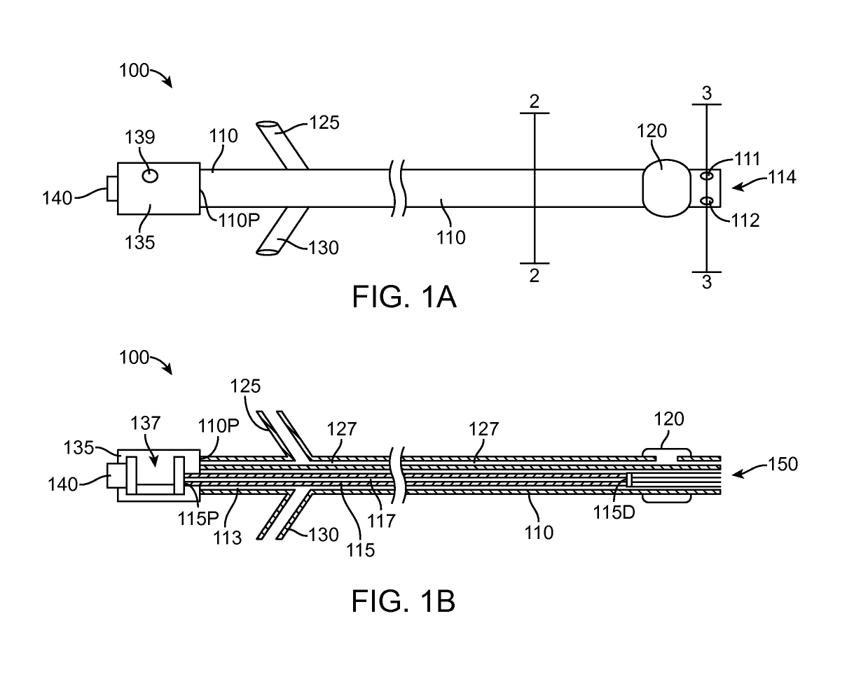

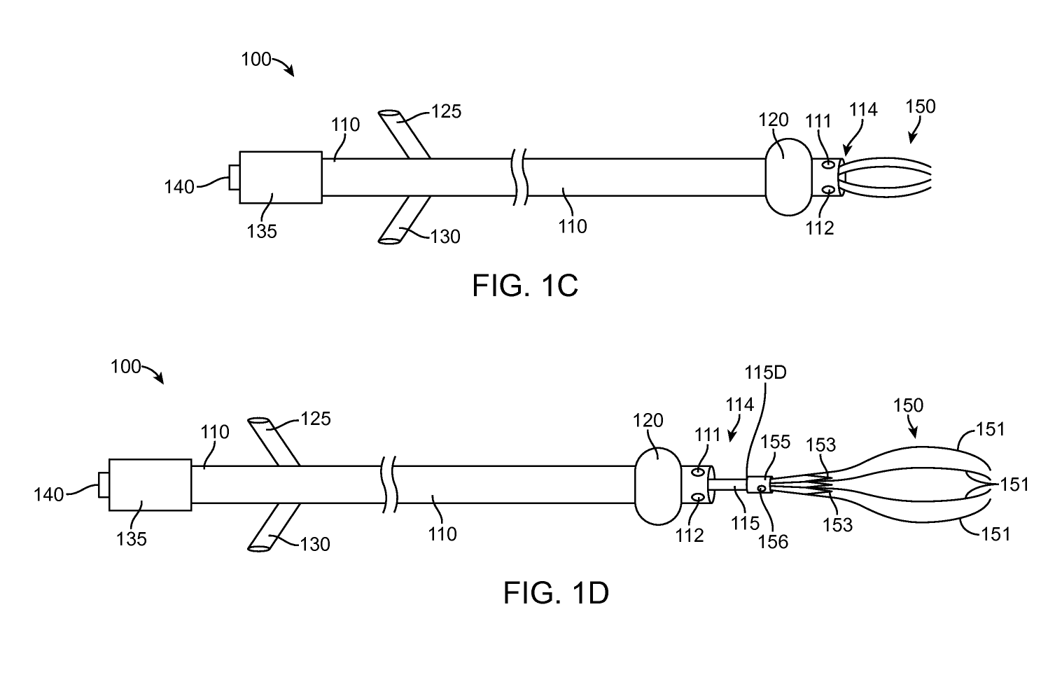

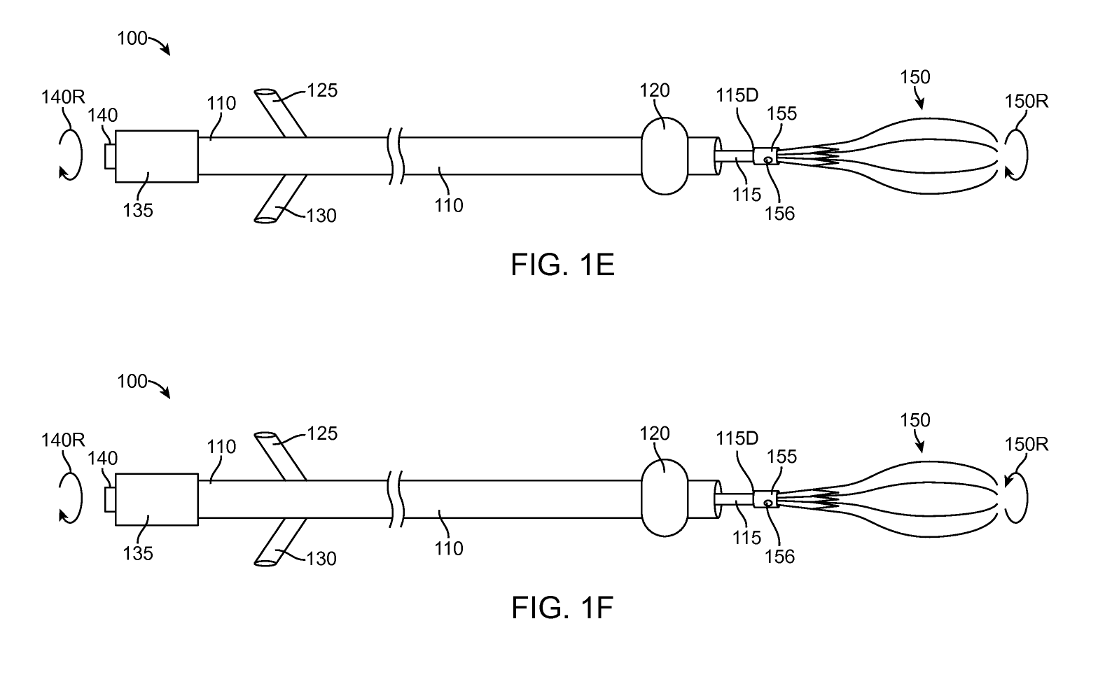

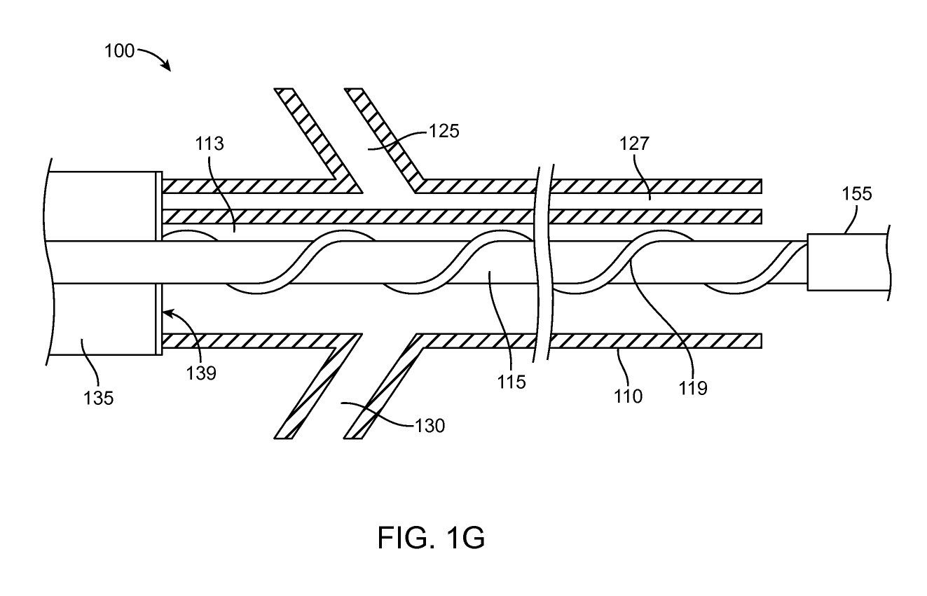

FIG. 1A shows a blood clot extraction device 100 according to an embodiment of the disclosure. FIG. 1B shows the blood clot extraction device 100 in cross-section. As shown in FIGS. 1A and 1B, the blood clot extraction device 100 comprises an elongate outer shaft 110, an elongate inner shaft 115 slidably disposed within the inner lumen 113 of the elongate outer shaft 110, a set of clot capture arms 150 coupled to the distal end 115D of the elongate inner shaft 115, and a proximal handle portion or hub 135 coupled to the proximal end 110P of the elongate outer shaft 110 and the proximal end 115P of the elongate inner shaft 110. The elongate outer shaft 110 has an open distal end 114 through which the set of clot capture arms 150 can extend through. The elongate outer shaft 110 and/or the elongate inner shaft 115 are typically flexible and rotatable such that they can be advanced and maneuvered through the vasculature to reach a clot. Different sections of the elongate outer shaft 110 may be comprised of several sections specifically suited for different anatomical structures. For example, the elongate outer shaft 110 may have different mechanical characteristics such as flexibility, torquablility, and unique shapes for ease of device navigation and positioning. The elongate inner shaft 115 disposed within the elongate outer shaft 110 may also serve to provide mechanical support to the elongate outer shaft 110. The outer diameter of the elongate outer shaft 110 may range from 1 mm to 8 mm, preferably 4 mm to 6 mm, and more preferably 2 mm to 5 mm. The elongate inner shaft 115 may have a working length in the range from 80 cm to 2,000 cm, preferably 100 cm to 175 cm, and more preferably 120 cm to 150 cm.

As shown in FIGS. 1A and 1B, the set of clot capture arms 150 are constrained within the inner lumen 113 at the distal portion of the elongate outer shaft 110. At or near the open distal end 114, the elongate outer shaft 110 may include a radiopaque marker 111 for use as a land mark to indicate the distal portion of the elongate outer shaft 110. The radiopaque marker 111 facilitates the location and navigation of the device 100 during a procedure and can also mechanically reinforce the distal end 114 of the elongate outer shaft 110 to reduce mechanical deformation. The elongate outer shaft 110 may further comprise a sensor element 112. The sensor element 112 may be an ultrasound or other pressure sensor for detecting the presence of nearby clots. The proximal handle portion or hub 135 may comprise an indicator light 139 coupled to the sensor element 112. The sensor element 112 may be mounted on the top of the outer diameter of the elongate outer shaft 110 with inward focal energy allowing for clot breakage while traveling towards the proximal end of the device facilitating clot removal and transportation during extraction.

The blood clot extraction device 100 may further comprise an expandable element 120 for anchoring the device 100 at a desired location in a blood vessel. The expandable element 120 may also be partially or completely expanded during advancement of the blood clot extraction device 100 to help guide the device 100 into a desired position. The expandable element 120 will typically be an inflatable balloon 120. As shown in FIG. 1B, the elongate outer shaft 110 further comprises an inflation lumen 127 open at the proximal portion of the elongate outer shaft 110 to an inflation port 125 and at the distal portion of the elongate outer shaft 110 to the inflatable balloon 120 so that it can be inflated or deflated. Also at the proximal portion of the elongate outer shaft 110, the inner lumen 113 of the elongate outer shaft 110 is open to an access port 130. Through port 130, fluid such as saline can be introduced into the inner lumen 113 and passed out through the open distal end 114. Also, captured blood clot can be extracted through the inner lumen 113 and the port 130 by the application of negative pressure, e.g., by a syringe. In some embodiments, for example as shown in FIG. 1B, the elongate inner tube 115 comprises an inner lumen 117 which may be used for the application of negative pressure, the application of suction, the advancement of a guide wire, or the introduction of chemical agents, drugs, or various solutions. In other embodiments, the elongate inner tube 115 has a solid core to provide more rigidity and torquablity to the elongate inner tube 115.

The proximal end 115P of the elongate inner tube 115 is coupled to the proximal handle portion or hub 135. The proximal handle portion or hub 135 may further comprise one or more ports for the introduction or removal of various fluids, the application of negative pressure or suction, the introduction of a guidewire through the hub 135 and into the lumen of the inner shaft 115 or the outer shaft 110, etc. The proximal handle portion or hub 135 may be configured to fit comfortably in the palm of a user. In many embodiments, for example as shown in FIG. 1B, the proximal handle portion or hub 135 may comprise a rotatable element 140 which can be rotated to rotate the elongate inner shaft 115 and a gear mechanism 137 to modify the rotation of the rotatable element 140 to a rotation of the elongate inner shaft 115. The elongate inner tube 115 can be translated proximally and distally from the proximal handle portion or hub 135.

As shown in FIG. 1C, the elongate inner tube 115 has been advanced in the distal direction such that the set of clot capture arms 150 have partially extend out through the open distal end 114 of the elongate outer tube 110. Further advancement of the elongate inner tube 115 in the distal direction pushes the set of clot capture arms 150 fully out of the elongate outer tube 110 as shown in FIG. 1D. As the set of clot capture arms 150 extend out of the elongate outer tube 110, the clot capture arms 150 open radially. Typically, the set of clot capture arms 150 will be biased to be in their radially open configuration shown in FIG. 1D. As shown in FIG. 1D, the set of clot capture arms 150 comprise a plurality of elongate arms 151 having radially inwardly curved distal portions. By being radially inwardly curved, the shape of the elongate arms 151 minimizes injury to the inner wall of the blood vessel where they open. Also, in cases of a bifurcating vessel, the radially inwardly curved shape can prevent one or more of the elongate arms from inadvertently passing into a branch vessel. The set of clot capture arms 150 further comprise connectors 153 which connect adjacent elongate arms 151 to one another at their proximal portions. The set of clot capture arms 150 are typically coupled to the distal end 115D of the elongate shaft 115 through a coupling element 155 which may further comprise a radiopaque marker 156 for facilitating and navigation of the set of clot capture arms 150. The set of clot capture arms 150 may be made of various materials, including stainless surgical steel or shape memory materials such as nitinol.

As discussed above, the elongate inner shaft 115 can be rotated. The rotation of the elongate inner shaft 115 rotates the set of clot capture arms 150. Typically, the elongate inner shaft 115 is rotated by rotation of the rotation element 140. The elongate inner shaft 115 will typically be able to transmit torque from the proximal end 115D of the shaft 115 to the distal end 115D. Rotation can occur in many ways. As shown in FIG. 1E, rotation of the rotation element 140 will rotate the elongate inner shaft 115 in the same direction. For example, rotation of the rotation element 140 in a clockwise direction 140R will rotate the inner shaft 115 and the set of clot capture arms 150 in the same clockwise direction 140R. In some embodiments, the gear mechanism 137 will have a gear ratio such that a single rotation of the rotation element 140 will rotate the elongate inner shaft 115 and the set of clot capture arms 150 less than or greater than a single time. For example, a single rotation of the rotation element 140 may lead to a quarter, a half, a three quarter, a 1.25x, a 1.5x, a 1.75x, a 2.0x, etc. rotation of the elongate inner shaft 115 and the set of capture arms 150.

The gear mechanism 137 may also change the direction of rotation. As shown in FIG. 1F, rotation of rotation element 140 in a clockwise direction 140R can cause the elongate inner shaft 115 and the set of clot capture arms 150 to rotate in a counter-clockwise direction 150R′. In some embodiments, for example as shown in FIG. 1G, the outer surface of the elongate inner shaft 115 comprises a thread 119 which will typically be a reverse thread. Rotation of the elongate inner shaft 115 in a counter-clockwise direction 150R′ will carry captured clot material proximally within the inner lumen 113 of the elongate outer shaft 110. This clot material will be diverted into port 130 for extraction. The inner lumen 113 of the elongate outer shaft 110 will be tightly sealed from the interior of the proximal handle portion or hub 135 with a fluid seal 139 to prevent any clot material from interfering with the gear mechanism 137 and the rotatability of the rotation element 140. The fluid seal 139 may comprise, for example, a collet. As shown in FIG. 1G, the elongate inner shaft 115 can extend through into the proximal handle portion or hub 135 through a port in the hub 135. The proximal end 115P can then couple with the rotation element 140 within the hub 135. In some embodiments, the elongate inner shaft 115 extends fully out of the hub 135 in the proximal direction such that the proximal portion of the elongate inner shaft 115 can be rotated directly. Again, this rotated proximal portion of the elongate inner shaft 115 can be fluidly isolated from the inner lumen 113 of the elongate outer shaft 110 with fluid seal 139.

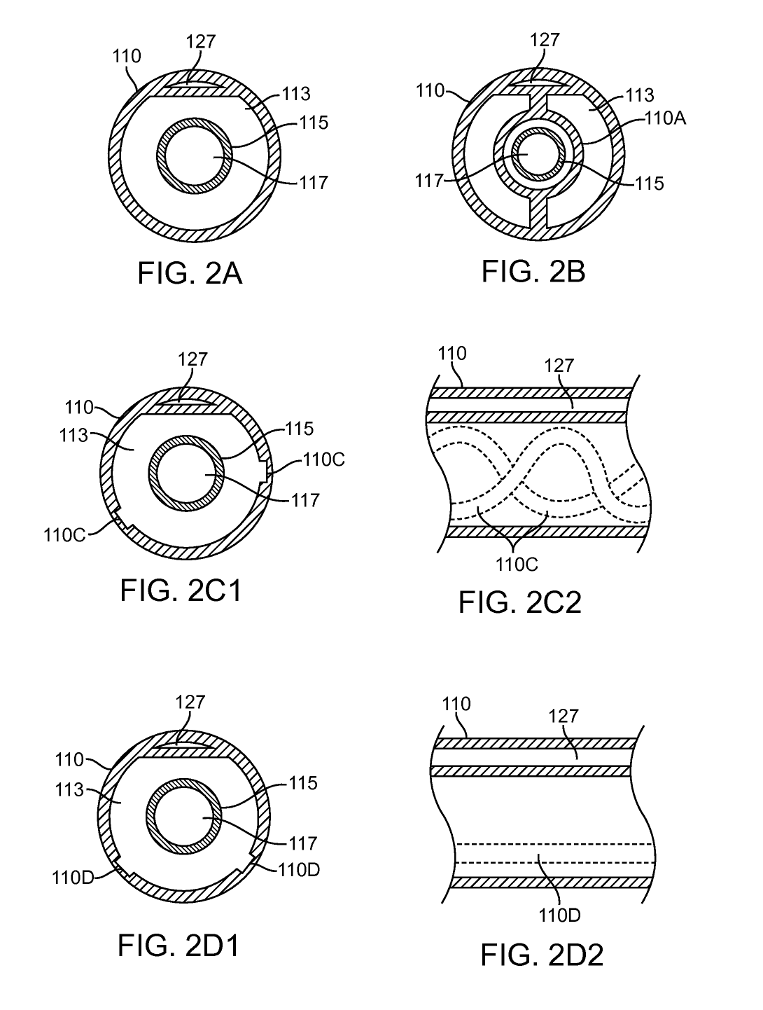

FIG. 2A is a cross-sectional view of the blood clot extraction device 100 taken along line 2 in FIG. 1A. As discussed above, the elongate outer shaft 110 comprises an inflation lumen 127 and an inner lumen 113. The inflation lumen 127 is isolated from the central inner lumen 113. The elongate inner shaft 115 is slidably disposed within the inner lumen 113. The elongate inner shaft 115 can have an inner lumen 117.

FIG. 2B is a cross-sectional view of a blood clot extraction device 100 according to another embodiment. In this embodiment, the elongate outer shaft 110 further comprises an inner tube 110A which houses the elongate inner shaft 115. The inner tube 110A isolates the elongate inner shaft 115 from the inner lumen 113, for example, to prevent captured clot material being extracted through inner lumen 113 from interfering with the movement of the elongate inner shaft 115.

The inner wall of the elongate outer tube 110 may also comprise various shapes and/or various structures to facilitate the extraction of captured clot material through the inner lumen 113. For example, as shown in FIGS. 2C1 and 2C2, the inner wall of the elongate outer tube 110 may comprise one or more spiral or serpentine grooves 110C. In another example, the inner wall of the elongate outer tube 110 may comprise one or more linear grooves 110D.

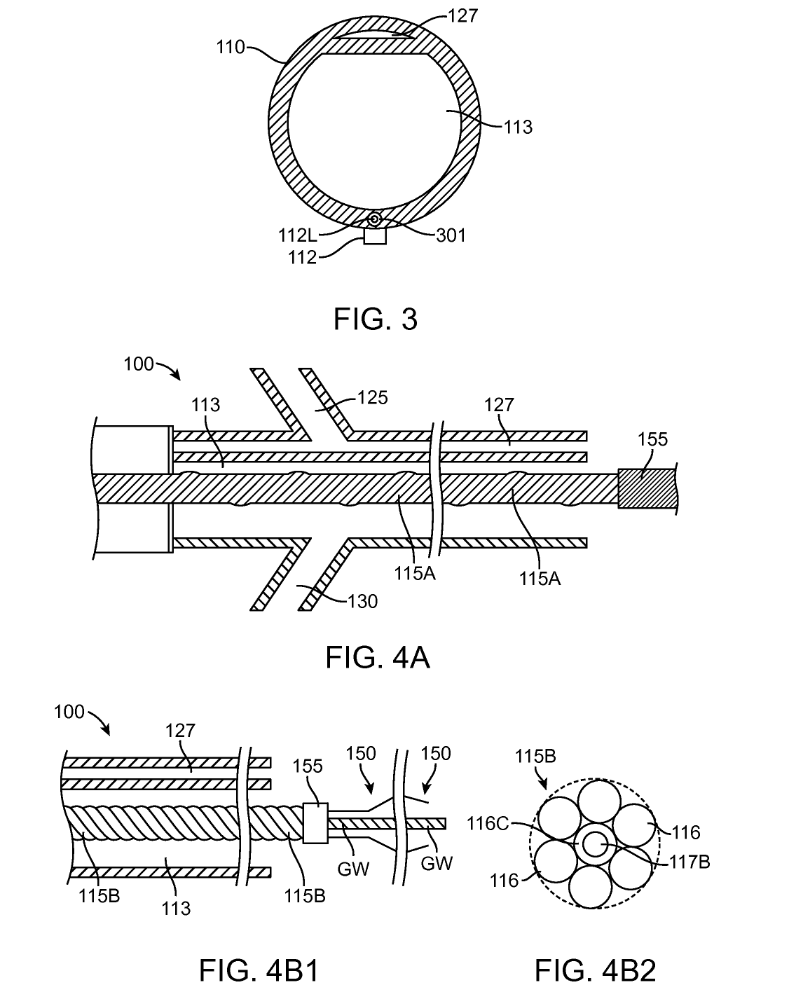

As discussed above, the elongate outer shaft 110 may include a sensor element 112 at or near the distal end 114 of the shaft 110. FIG. 3 is a cross-sectional view of the blood clot extraction device 100 taken across line 3 in FIG. 1A. As shown in FIG. 3, the elongate outer shaft 110 may further comprise a sensor lead lumen 301 so that an electrical lead 112L from the sensor element 112 can be isolated and shielded from the inner lumen 113 of the shaft 110 as well as the exterior of the shaft 110. The sensor lead lumen 301 may extend to the proximal end 110P of the elongate outer shaft 110 so that the electrical lead 112L from the sensor 301 can coupled to the indicator light 139 on the proximal handle portion or hub 135.

As discussed above, the inner shaft of the blood clot extraction device 100 may come in many forms. As shown in FIG. 4A, the blood clot extraction device 100 having a threaded inner shaft 115A with a solid core. By having a solid core, the inner shaft 115A may be more rigid and torquable. As shown in FIGS. 4B1 and 4B2, the blood clot extraction device 100 may comprise an elongate inner shaft 115B that comprises a plurality of wound cables 116. The cables 116 may be wound in a spiral or twisted fashion over the central cable 116C to form a reverse thread. This reverse thread allows rotation of the inner shaft 115A to carry clot material proximally over the inner shaft 115A. The central cable 116C defines an inner lumen 117B. The inner lumen 117B can have the same function as the inner lumen of the elongate inner shaft 115 described above. As shown in FIG. 4B1, a guide wire GW can be disposed within the inner lumen 117B. The blood clot extraction device 100, including the elongate inner shaft 117B, can be advanced over the guide wire GW.

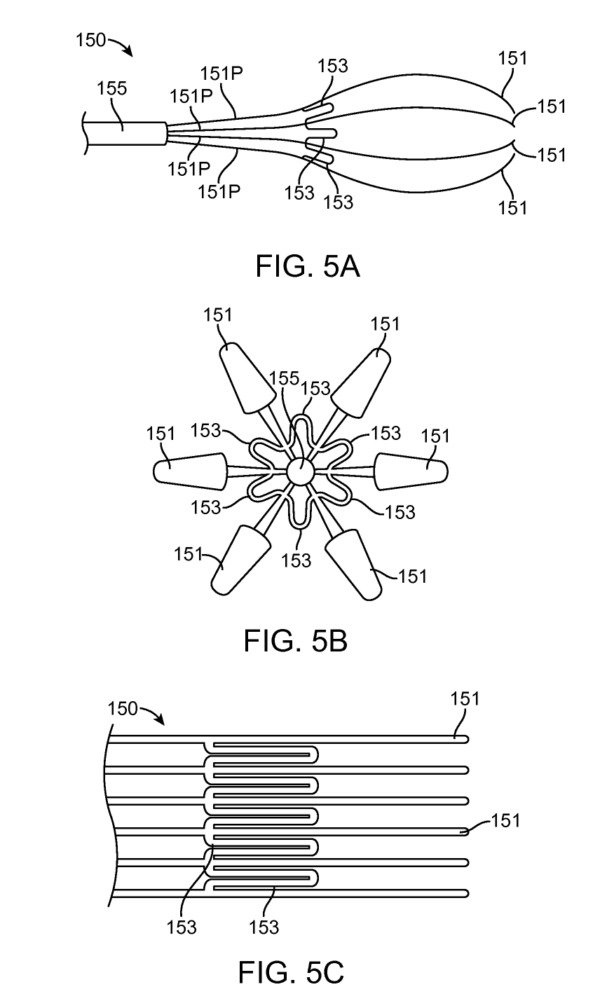

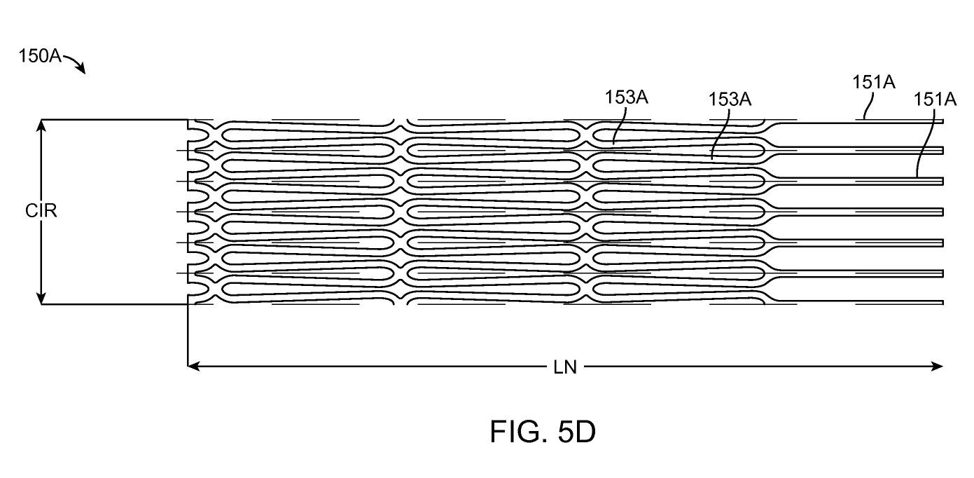

FIG. 5A shows a magnified side view of the set of clot capture arms 150 according to many embodiments, FIG. 5B shows an end view of the same set of clot capture arms 150, and FIG. 5C shows a side view of the same set of clot capture arms 150 in a flat, rolled out configuration. As discussed above, the set of clot capture arms 150 comprises a plurality of elongate, radially inwardly curved arms 151. Adjacent arms 151 are coupled together at their proximal portions 151P with connectors 153. There may be connectors 153 for all of the arms 151 or for only a select number of them. The connectors 153 will typically be U-shaped, with the open end of the U-shaped connector 153 facing proximally and the curved, closed end of the U-shaped connector 153 facing distally. The U-shaped connectors 153 can help to maintain the arms 151 in a desired position relative to one another. As shown in FIG. 5B, the curved arms 151 are arranged in pairs. The pairs of arms 151 will typically be diametrically opposed. As shown in FIGS. 5A to 5C, there are three pairs of arms 151. In other embodiments, other numbers of arms, such as one, two, four, five, or more, may instead be provided. In its fully radially open configuration, the set of clot capture arms 150 may have a minimum diameter at its proximal section of 2 mm, a maximum diameter at its distal section of 8 mm, a total length of 17 mm, and a length of 8 mm of its proximal portion 151P.

FIG. 5D shows a side view of a set of blood clot capture arms 150A rolled out and flattened according to another embodiment. The blood clot capture arms 150A comprise a plurality of struts arranged to form a plurality of elongate arms 151A coupled to one another with connectors 153A. The set of clot capture arms 150A have a length LN and a circumference CIR. For example, the struts may have a strut width of 0.011 inches, the length LN of the set of clot capture arms 150A may be 1.256 inches, the circumference CIR of the set of clot capture arms 150A may be 0.295 inches, and the set of blood clot capture arms 150A may have an inner diameter of 0.070 inches and an outer diameter of 0.094 inches when rolled into a cylindrical or tubular configuration.

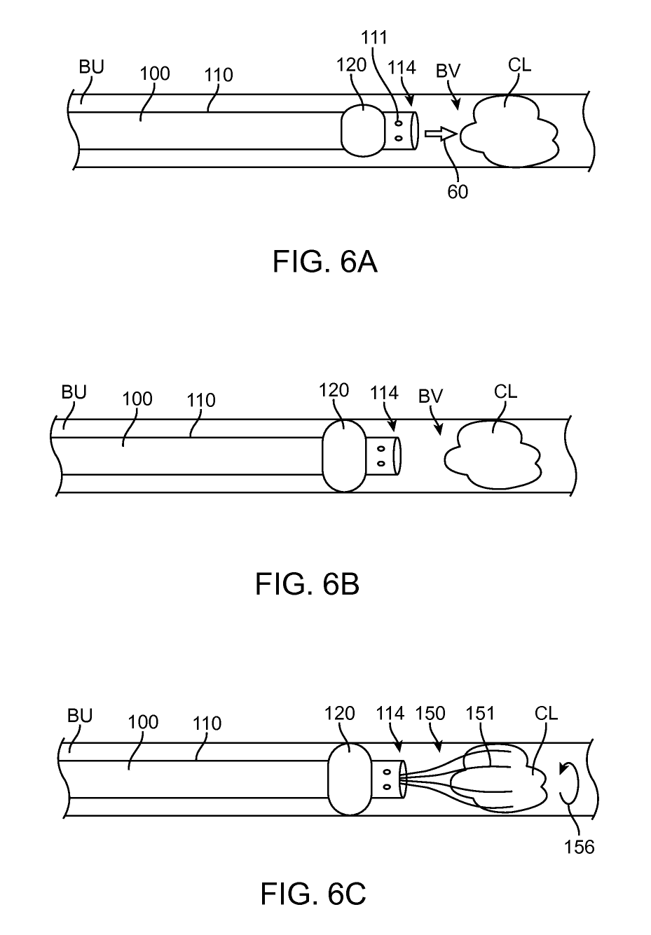

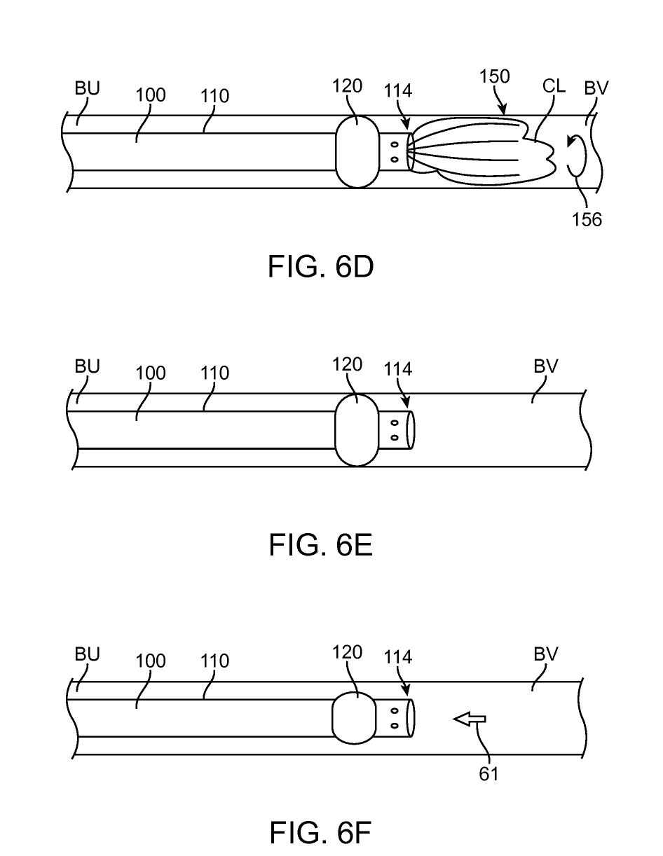

FIGS. 6A to 6F show an exemplary method of extracting a blood clot CL in a blood vessel BV with the blood clot extraction device 100. The blood vessel BV may be a pulmonary artery or a peripheral vein. The blood clot extraction device 100 may be initially introduced into the vasculature of a patient through the groin or jugular vein. The user or operator can navigate the device 100 toward an occluded blood vessel BV with the assistance of the radiopaque marker 111 and/or the partial expansion of the expandable element 120.

As shown in FIG. 6A, the blood clot extraction device 100 is distally advanced in the blood vessel BV in a distal direction indicated by arrow 60 to be near the site of a blood clot CL. The extraction device 100 may be advanced under fluoroscopy with the assistance of the radiopaque marker 111. As shown in FIG. 6B, the inflation balloon 120 is expanded to anchor the extraction device 100 in the blood vessel. As shown in FIG. 6C, the set of clot capture arms 150 is advanced out from the open distal end 114 of the elongate outer shaft 110. The set of clot capture arms 150 are often configured to minimize vessel trauma and/or to self adjust to the vessel size as deployed and retracted, for example by having a radially inwardly curved shape at their distal portions and by their gradual radial expansion or contraction in unison with axial advancement or retraction, respectively (i.e., the set of clot capture arms 150 may expand to lesser or greater radial sizes based on their level of advancement out of the elongate outer shaft 110). At least a portion of the blood clot CL will be captured by the elongate arms 151 of the set of clot capture arms 150. The set of clot capture arms 150 may be rotated in a direction indicated by arrow 156 to facilitate such capture. Rotation of the set of clot capture arms 150 and/or advancement of the set of clot capture arms 150 into the clot CL can also serve to soften or break apart at least a portion of the blood clot CL. For instance, advancement of the elongate arms 151 into the blood clot CL may cut portions of the blood clot CL. In some embodiments, it may be desired to destroy or break at least a portion of the clot CL with the rotation or advancement of elongate arms 151. The clot material will then be captured via suction or the application of a negative pressure through the inner lumen 113 of the elongate outer shaft 110 or the inner lumen 117 of the elongate inner shaft 115.

As shown by FIG. 6D, with the blood clot CL captured the set of clot capture arms 150 may be retracted back into the elongate outer shaft 110. As the set of clot capture arms 150 are retracted, they gradually close into a collapsed configuration, reducing the profile of the captured blood clot CL as well. The reduction in profile of the blood clot CL can allow the clot CL to be captured within the elongate outer shaft 110. As discussed above, the elongate inner shaft 110 may be threaded. Rotation of the elongate inner shaft 115, for example in the counter-clockwise direction indicated by arrow 156 where the elongate inner shaft 115 comprises a reverse thread, can carry portions of the clot CL proximally through the inner lumen 113 of the elongate outer shaft 110 to further facilitate the capture of the clot CL within the clot extraction device 100. As shown in FIG. 6E, the clot CL can be fully captured into the inner lumen 113 of the elongate outer shaft 110 as the set of clot capture arms 150 is retracted fully into the inner lumen 113 of the elongate outer shaft 110. As shown in FIG. 6F, the anchoring inflation balloon 120 can then be deflated to de-anchor the device 100, and the device 100 can be retracted proximally in a direction indicated by arrow 61 to remove the device 100 from the blood vessel BV.

While the device 100 is described above for use to remove a blood clot from a pulmonary artery or peripheral vein, the device 100 may also be used for other indications and anatomies including the removal of kidney stones, sinus blockages, outer and inner ear blockages, coronary blockages, heart valve blockages, blockages in the neurovasculature, spinal calcifications, etc.

While preferred embodiments of the present disclosure have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

What is claimed is:

1. A device for capturing clot in a blood vessel, the device comprising:
an outer shaft;
an inner shaft slidably disposed in a lumen of the outer shaft; and
a plurality of clot capture arms secured to a distal end of the inner shaft, wherein at least some of the arms have radially inwardly curved distal portions, and wherein the arms are configured to open as the distal portions are advanced from the outer shaft and close as the distal portion is retracted into the outer shaft.

2. The device of claim 1, wherein the inner shaft is rotatable within the lumen of the outer shaft.

3. The device of claim 2, further comprising a proximal hub coupled to a proximal end of the inner shaft, the proximal hub comprising a rotatable element rotatable in a first direction, wherein rotating the rotatable element in the first direction causes the inner shaft to rotate in a second direction opposite the first direction.

4. The device of claim 2, wherein the inner shaft is threaded such that rotation of the inner shaft causes a thread of the inner shaft to carry at least a portion of a captured clot in a proximal direction to remove the clot.

5. The device of claim 4, wherein the thread of the tubular inner shaft comprises a reverse thread.

6. The device of claim 1, wherein the plurality of arms are biased to open.

7. The device of claim 1, wherein the plurality of arms are shaped to reduce vessel trauma when opened and advanced from the outer shaft.

8. The device of claim 1, wherein the plurality of arms comprises at least one pair of diametrically opposed arms.

9. The device of claim 1, wherein the plurality of arms comprise at least one connector coupling an arm of the plurality of arms to an adjacent arm.

10. The device of claim 10, wherein the at least one connector couples a proximal portion of the arm of the plurality of arms to a proximal portion of the adjacent arm.

11. The device of claim 10, wherein the connector comprises a U-shaped connector.

12. The device of claim 1, further comprising an expandable element adjacent the distal end of the outer shaft, the expandable element being expandable to anchor the outer shaft within the blood vessel near the clot.

13. The device of claim 1, further comprising a hub coupled to a proximal end of the outer shaft and a proximal end of the inner shaft.

14. The device of claim 13, wherein the hub is configured to divert captured and removed clot away from the proximal end of the outer shaft and the proximal end of the inner shaft.

15. The device of claim 13, wherein the inner shaft comprises a lumen, and wherein the device further comprises a suction element coupled to the hub, the suction element configured to apply suction to remove clot from at least one of the lumen of the outer shaft and the lumen of the inner shaft.

16. A method for treating a pulmonary embolism by capturing clot in a pulmonary artery or a peripheral vein, the method comprising:
introducing a catheter to a location in the pulmonary artery or a peripheral vein proximal of the clot;
advancing at least distal portions of a plurality of clot capture arms out from an open end of the catheter to surround at least a portion of the clot; and
retracting the distal portions of the plurality of clot capture arms back into the catheter to capture the portion of the clot.

17. The method of claim 16, further comprising expanding an expandable element near the open end of the outer shaft to anchor the outer shaft near the clot in the pulmonary artery or the peripheral vein.

18. The method of claim 16, further comprising breaking the portion of the clot.

19. The method of claim 18, wherein breaking the portion of the clot comprises rotating the plurality of clot capture arms against the clot.

20. The method of claim 18, wherein breaking the portion of the clot comprises distally advancing the plurality of clot capture arms against the clot.

21. The method of claim 16, further comprising rotating an inner shaft slidably disposed within the outer shaft, the inner shaft having a distal end coupled to a proximal end of the plurality of clot capture arms.

22. The method of claim 21, wherein rotating the inner shaft causes a thread of the inner shaft to carry the portion of the clot in a proximal direction within the outer shaft to remove the portion of the clot.

23. The method of claim 21, wherein rotating the inner shaft comprises rotating a rotatable element of a hub coupled to a proximal end of the inner shaft in a first direction to rotate the distal end of the inner shaft in a second direction opposite the first direction.

24. The method of claim 16, wherein advancing the plurality of clot capture arms from the open end of the outer shaft comprises opening the clot capture arms as the arms are advanced from the open end of the outer shaft.

25. The method of claim 16, further comprising applying suction through at least one of an inner lumen of the outer shaft and an inner lumen of the inner shaft to remove the captured portion of clot.
